(19) United States
(12) Patent Application Publication
(10) Pub. No.: US 2025/0099538 A1
(43) Pub. Date: Mar. 27, 2025

(54) METHODS OF TREATING NEURODEGENERATIVE DISEASES

(71) Applicant: Academia Sinica, Taipei City (TW)

(72) Inventors: Che-Kun James SHEN, Taipei City (TW); Shih-Ling HUANG, Yunlin County (TW); Chun-Hao HUNG, Tainan City (TW)

(21) Appl. No.: 18/686,862

(22) PCT Filed: Aug. 26, 2022
(86) PCT No.: PCT/US2022/041643

§ 371 Date: Feb. 26, 2024

Related U.S. Application Data
(60) Provisional application No. 63/237,574, filed on Aug. 27, 2021.

(51) Int. Cl. A61K 38/17; A61K 35/28; A61P 25/28; C07K 14/47

(52) U.S. Cl. CPC A61K 38/1709; A61K 35/28; A61P 25/28; C07K 14/4705

(57) ABSTRACT

Disclosed herein are methods of preventing or treating ALS by use of a modified EKLF polypeptide, a modified nucleic acid encoding the modified EKLF polypeptide, or modified bone marrow cells comprising the modified nucleic acid. According to embodiments of the present disclosure, the modified EKLF polypeptide comprises an amino acid modification that confers reduced sumoylation in a wild-type EKLF polypeptide.

### CROSS-REFERENCE TO RELATED APPLICATIONS

This application is a national stage entry under 35 U.S.C. 371 from International Patent Application Serial No. PCT/US2022/41643, filed Aug. 26, 2022, and published on Mar. 2, 2023, designated the United States. The PCT application claims priority from U.S. Provisional Application No. 63/237,574, filed Aug. 27, 2021; the contents of these applications are incorporated herein by reference in their entireties.

### SEQUENCE LISTING XML

The present application is being filed along with a Sequence Listing XML in electronic format. The Sequence Listing XML is provided as an XML file entitled P4216-US_SEQ AF, created Aug. 28, 2024, which is 22 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to methods of treating neurodegenerative disease by use of a modified Erythroid Kruppel-like factor (EKLF) polypeptide, which comprises an amino acid modification that confers reduced sumoylation in a wild-type EKLF polypeptide.

### 2. Description of Related Art

Amyotrophic lateral sclerosis (ALS: also known as Lou Gehrig's disease) is a progressive nervous system disease that affects nerve cells in the brain and spinal cord that control voluntary muscle movements, such as walking and talking. ALS is the most common type of motor neuron diseases. Early symptoms of ALS include stiff muscles, muscle twitches. and gradual increasing weakness and muscle wasting. As the disease progresses. ALS causes a variety of complications, including breathing problems, speaking problems and eating problems. Half of ALS patients develop at least mild difficulties with thinking and behavior, and about 15% of the ALS patients develop frontotemporal dementia. ALS eventually causes paralysis and early death, usually from respiratory failure.

Two drugs are approved by the U.S. Food and Drug Administration (FDA) for treating ALS, including Riluzole and Edaravone. The administration of Riluzole increases the life expectancy of ALS patients by three to six months. Edaravone is known to reduce the decline in daily function of early-stage ALS patients. However, both treatments only slow the progression of ALS symptoms and prevent the complications associated with ALS, but can't reverse the damage of ALS. Up to the present day, there is no cure for ALS.

In view of the foregoing, there is a continuing interest in developing a novel method for treating ALS thereby improving the life quality and/or lifespan of ALS patients.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later

The present disclosure provides a method of preventing and/or treating a neurodegenerative disease in a subject. The method comprises administering to the subject an effective amount of a modified EKLF polypeptide, a modified nucleic acid encoding the modified EKLF polypeptide, or modified bone marrow cells comprising the modified nucleic acid. According to some embodiments of the present disclosure, the modified EKLF polypeptide comprises an amino acid modification that confers reduced sumoylation in a wild-type EKLF polypeptide.

According to some embodiments, the subject is a human, in which the amino acid modification is a substitution of lysine residue with arginine residue at the amino acid position 54 of a wild-type EKLF polypeptide of SEQ ID NO: 3. In these embodiments, the modified EKLF polypeptide comprises the amino acid sequence of SEQ ID NO: 10.

According to certain embodiments, the subject is a mouse, in which the amino acid modification is a substitution of lysine residue with arginine residue at the amino acid position 74 of a wild-type EKLF polypeptide of SEQ ID NO: 1. In these embodiments, the modified EKLF polypeptide comprises the amino acid sequence of SEQ ID NO: 11.

According to some embodiments of the present disclosure, the modified bone marrow cells comprise modified hematopoietic stem cells (HSCs), modified hematopoietic stem and progenitor cells (HSPCs), or a combination thereof.

According to certain embodiments, the modified bone marrow cells are autologous to the subject. According to alternative embodiments, the modified bone marrow cells are allogeneic or xenogeneic to the subject; in the alternative embodiments, the present method further comprises the step of exposing the subject to a gamma irradiation or administering to the subject an immunosuppressant prior to the administration of the modified bone marrow cells.

The modified polypeptide, modified nucleic acid or modified bone marrow cells may be administered to the subject via any appropriate route, for example, intravenous, intraperitoneal, intraarterial or intraspinal injection. Preferably, the modified polypeptide, modified nucleic acid or modified bone marrow cells is/are intravenously injected to the subject.

Non-limiting examples of neurodegenerative disease treatable with the present method includes amyotrophic lateral sclerosis (ALS), Lewy Body Dementia (LBD), Parkinson's disease (PD), Alzheimer's disease (AD), multiple sclerosis (MS), or Huntington's disease (HD). According to some exemplary embodiments, the neurodegenerative disease is ALS.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A is a histogram depicting the behavioral comparison of genetic-crossed mice in rotarod test. N>5, *, p<0.05; **, p<0.01. TDP-43(+/+); Eklf(+/+): wild-type (WT) mouse having a WT Eklf gene and a WT TAR DNA binding protein (Tardbp) gene; TDP-43(N390D/+); Eklf (+/+): ALS mouse having a mutant (N390D) Tardbp gene and a WT Eklf gene; TDP-43(+/+); Eklf (K74R/K74R): Eklf (K74R) mice having a WT Tardbp gene and a mutant Eklf (K74R) gene; TDP-43(N390D/+); Eklf (K74R/K74R): N390D/+/Eklf (K74R) mouse having a mutant (N390D) Tardbp gene and a mutant Eklf (K74R) gene.

FIG. 1B is a histogram depicting the result of rotarod test of mice receiving specified bone marrow transplantation. N>5, *, p<0.05, **, p<0.01. WT: wild-type mouse having a WT Eklf gene and a WT Tardbp gene; ALS: ALS mouse having a mutant (N390D) Tardbp gene and a WT Eklf gene; Eklf (K74R): Eklf (K74R) mice having a WT Tardbp gene and a mutant Eklf (K74R) gene.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “bone marrow cells” refers to cells at various differentiation sages that exist in the bone marrow, including cells of hematopoietic origin (such as hematopoietic repopulating cells, hematopoietic stem cells, and hematopoietic stem and progenitor cells), and cells derived from bone marrow (such as endothelial cells, mesenchymal cells, bone cells, neural cells, and supporting cells (also known as stromal cells)).

The term “autologous” indicates the origin of a bio-material (e.g., bone marrow cells). More specifically, the term “autologous” as used herein refers to a bio-material (e.g., bone marrow cells) derived from an individual and re-introduced (with or without modification) to the same individual. For example, the term “autologous transplantation” refers to a transplantation, in which the donor and recipient of the transplant are the same individual. Such procedures are advantageous because they overcome the immunological barrier which otherwise results in rejection.

The term “allogeneic” as used herein refers to a bio-material (e.g., bone marrow cells) derived from an individual, and introduced (with or without modification) to another individual of the same species. For example, the term “allogeneic transplantation” refers to a transplantation, in which the donor and recipient are different individuals of the same species.

The term “xenogeneic” as used herein refers to a bio-material (e.g., bone marrow cells) derived from an individual of one species, and introduced (with or without modification) to an individual of another species. For example, the term “allogeneic transplantation” refers to a transplantation, in which the donor and recipient are different species.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

The term “polypeptide” refers to a polymer of amino acids without regard to the length of the polymer; thus, “peptides,” “oligopeptides,” and “proteins” are included within the definition of polypeptide and used interchangeably herein. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. Throughout the present disclosure, the positions of any specified amino acid residues within a polypeptide are numbered starting from the N terminus of the polypeptide. When amino acids are not designated as either D- or L-amino acids, the amino acid is either an L-amino acid or could be either a D- or L-amino acid, unless the context requires a particular isomer. Further, the notation used herein for the polypeptide amino acid residues are those abbreviations commonly used in the art.

The term “administered”, “administering” or “administration” are used interchangeably herein to refer a mode of delivery, including, without limitation, intravenously. intraperitoneally, intraarterially or intraspinally delivering an agent (e.g., the modified EKLF polypeptide, modified nucleic acid, or modified bone marrow cells) of the present invention. In some embodiments, the modified bone marrow cells are intravenously injected to a subject in need thereof (e.g., a subject having or suspected of having ALS).

As used herein, the term “treat,” “treating” and “treatment” are interchangeable, and encompasses partially or completely ameliorating, mitigating and/or managing a symptom, a secondary disorder or a condition associated with ALS. The term “treating” as used herein refers to application or administration of the modified EKLF polypeptide, modified nucleic acid, or modified bone marrow cells of the present disclosure to a subject, who has a symptom, a secondary disorder or a condition associated with ALS, with the purpose to partially or completely alleviate, ameliorate, relieve, delay onset of, inhibit progression of, reduce severity of, and/or reduce incidence of one or more symptoms, secondary disorders or features associated with ALS. Symptoms, secondary disorders, and/or conditions associated with ALS include, but are not limited to, stiff muscles, muscle twitches, muscle weakness, muscle wasting, difficulty walking, tripping and falling, hand weakness or clumsiness, slurred speech, trouble swallowing, inappropriate behavior (e.g., inappropriate crying, laughing or yawning), cognitive problems, and behavioral changes. Treatment is generally “effective” if one or more symptoms or clinical markers are reduced as that term is defined herein. Alternatively, a treatment is “effective” if the progression of a symptom, disorder or condition is reduced or halted.

The term “prevent” or “preventing” and as used herein are interchangeable, and refers to the prophylactic treatment of a subject who is at risk of developing a symptom, a secondary disorder or a condition associated with ALS, so as to decrease the probability that the subject will develop the symptom, secondary disorder or condition. Specifically, the term “prevent” or “preventing” refers to inhibit the occurrence of a symptom, a secondary disorder or a condition associated with ALS, that is to reduce the incidence or the frequency of occurrence of the symptom, secondary disorder or condition. The term “prevent” or “preventing” as used herein referring to the modified EKLF polypeptide, modified nucleic acid and/or modified bone marrow cells does not mean or imply that use of the modified EKLF polypeptide, modified nucleic acid and/or modified bone marrow cells will provide a guarantee that the symptom, secondary disorder or condition of ALS will never occur, but rather that the modified EKLF polypeptide. modified nucleic acid and/or modified bone marrow cells will inhibit the occurrence of the symptom, secondary disorder or condition of ALS, and that the incidence and/or frequency of the symptom, secondary disorder or condition of ALS will be reduced.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives Effective amount may be expressed, for example, as grams, milligrams or micrograms; as milligrams per kilogram of body weight (mg/Kg); or as cell numbers of body weight (cells/Kg). Persons having ordinary skills could calculate the human equivalent dose (HED) for the medicament (such as the present modified EKLF polypeptide, the modified nucleic acid or the modified bone marrow cells) based on the doses determined from animal models. For example, one may follow the guidance for industry published by U.S. Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to a mammal including the human species that is treatable with the modified EKLF polypeptide, modified nucleic acid, modified bone marrow cells and/or method of the present invention. The term “subject” is intended to refer to both the male and female gender unless one gender is specifically indicated.

### II. Description of the Invention

EKLF, also named KLF1, is a Kruppel-like factor expressed in a range of blood cells, including erythrocytes, megakaryocytes, T cells, NK cells, as well as in various hematopoietic progenitors, including common myeloid progenitor (CMP), megakaryocyte-erythroid progenitor (MEP), and granulocyte-macrophage progenitor (GMP). EKLF can positively or negatively regulate transcription through binding of its zinc finger domain to the CACCC motif of the regulatory regions of a diverse array of genes. EKLF regulates erythropoiesis, and differentiation of MEP to megakaryocytes and erythrocytes, as well as of monocytes to macrophages. Most recently, it has also been shown that EKLF is expressed in long-term hematopoietic stem cells (LT-HSC) and regulates their differentiation.

The present disclosure is based, at least in part, on the discovery that a single amino acid substitution from a lysine (K) residue to an arginine (R) residue at the sumoylation site of EKLF would ameliorate or alleviate the symptoms associated with ALS in mouse models. According to some embodiments of the present disclosure, compared to ALS control mice (i.e., N390D/+ mice), the ALS mice having a modified EKLF allele (i.e., N390D/+/Eklf (K74R) mice) exhibit better exercise ability and learning memory. According to some embodiments of the present disclosure, the transplantation of bone marrows (i.e., bone marrow transplantation. BMT) from Eklf (K74R) mice to ALS mice improves the exercise ability and learning memory of ALS mice. According to certain examples, low chimerism (<20%) of the Eklf (K74R) blood cells (i.e., the blood cells derived from the Eklf (K74R) mice) in the peripheral blood of the recipient mice is sufficient to provide a therapeutic effect on ALS. Thus, the introduction of Eklf (K74R) mutation provides a potential means to prevent and/or treat ALS.

II-(1) Methods of Preventing and/or Treating ALS by Modified EKLF Polypeptide or Nucleic Acid Encoding the Same

The first aspect of the present disclosure is directed to a method of preventing and/or treating ALS in a subject. The method comprises administering to the subject an effective amount of a modified EKLF polypeptide or a modified nucleic acid encoding the same. According to some embodiments of the present disclosure, the modified EKLF polypeptide comprises an amino acid modification at the sumoylation site that confers reduced sumoy lation in the modified EKLF polypeptide.

In some embodiments, the modified EKLF polypeptide comprises an amino acid modification as compared to a wild-type mouse EKLF polypeptide (SEQ ID NO: 1). In some embodiments, the modified EKLF polypeptide comprises an amino acid modification as compared to a wild-type rat EKLF polypeptide (SEQ ID NO: 2). In some embodiments, the modified EKLF polypeptide comprises an amino acid modification as compared to a wild-type human EKLF polypeptide (SEQ ID NO: 3). In some embodiments, the modified EKLF polypeptide comprises an amino acid modification as compared to a wild-type chimpanzee EKLF polypeptide (SEQ ID NO: 4). In some embodiments, the modified EKLF polypeptide comprises an amino acid modification as compared to a wild-type rhesus monkey EKLF polypeptide (SEQ ID NO: 5). In some embodiments, the modified EKLF polypeptide comprises an amino acid modification as compared to a wild-type dog EKLF polypeptide (SEQ ID NO: 6). In some embodiments, the modified EKLF polypeptide comprises an amino acid modification as compared to a wild-type cattle EKLF polypeptide (SEQ ID NO: 7).

According to certain embodiments, the subject is a human having the Eklf gene of SEQ ID NO: 8 that encodes the wild-type human EKLF polypeptide of SEQ ID NO: 3; in these embodiments, the modified EKLF polypeptide comprises the amino acid sequence of SEQ ID NO: 10, which, as compared to the wild-type human EKLF polypeptide of SEQ ID NO: 3, has a substitution of lysine residue with arginine residue at residue 54 (i.e., a K54R substitution).

According to certain embodiments, the subject is a mouse having the Eklf gene of SEQ ID NO: 9 that encodes the wild-type mouse EKLF polypeptide of SEQ ID NO: 1; in these embodiments, the modified EKLF polypeptide comprises the amino acid sequence of SEQ ID NO: 11, which, as compared to the wild-type mouse EKLF polypeptide of SEQ ID NO: 1. has a substitution of lysine residue with arginine residue at residue 74 (i.e., a K74R substitution).

The present modified EKLF polypeptide or the nucleic acid encoding the modified EKLF polypeptide may be administered to the subject via a suitable route in accordance with intended purposes, for example, via intravenous, intraperitoneal, intraarterial or intraspinal injection.

The present modified EKLF polypeptide may be produced by conventional recombinant technology. For example, a nucleic acid comprising a coding sequence for the modified EKLF polypeptide may be prepared using PCR techniques, or any other method or procedure known to one skilled in the art. The nucleic acid molecules thus obtained may be inserted into a suitable expression vector to enable the expression of the encoded recombinant protein in a suitable host cell. In some embodiments, the expression vector may include additional sequences, which render this vector suitable for replication and integration in prokaryotes or eukaryotes. Alternatively or in addition, the expression vector may comprise transcription and translation initiation sequences (e.g., promoters or enhancers) and transcription and translation terminators (e.g., polyadenylation signals). Exemplary expression vectors include, but are not limited to, bacterial expression vector, yeast expression vector, baculoviral expression vector, and mammalian expression vector. Any of the nucleic acids coding for the present modified EKLF polypeptide, a vector (such as an expression vector) comprising the nucleic acid, and host cells comprising the vector are also within the scope of the present disclosure.

A variety of prokaryotic or eukaryotic cells can be used as the host-expression system to express the present modified EKLF polypeptide. Examples of the expression systems include, but are not limited to, microorganisms, such as bacteria, yeast cell, plant cell, eukaryotic cell (e.g., mammalian cell or CHO cell), etc. Methods for transducing the expression vector into the host-expression system are known by a skilled artisan, e.g., stable or transient transfection, lipofection, electroporation and infection with recombinant viral vectors.

II-(2). Methods of Preventing and/or Treating ALS via Bone Marrow Transplantation

The second aspect of the present disclosure pertains to a method of preventing and/or treating ALS in a mouse subject. The method comprises the steps of,
(a) isolating the bone marrow cells from the Eklf (K74R) mouse of the present disclosure; and
(b) transferring the bone marrow cells of step (a) to the mouse subject thereby alleviating or ameliorating the symptoms associated with ALS.

The Eklf (K74R) mouse is generated by the method described in U.S. Pat. No. 10,414,809. Hence, the detail description is omitted for the sake of brevity.

According to certain embodiments, the transfer of the bone marrow cells improves the exercise behavior and learning memory in the mouse subject.

According to some embodiments of the present disclosure, the Eklf (K74R) mouse comprises a modified Eklf gene, which encodes a modified EKLF polypeptide comprising the amino acid sequence of SEQ ID NO: 11. As described above, the modified EKLF polypeptide of SEQ ID NO: 11 is characterized by having a substitution of lysine residue with arginine residue at residue 74 (i.e., a K74R substitution) as compared to the wild-type mouse EKLF polypeptide of SEQ ID NO: 1.

According to certain embodiments, about 1×10⁴ to 1×10⁸ of the bone marrow cells isolated from the present Eklf (K74R) mouse are transferred to the mouse subject. Preferably, about 1×10⁵ to 1×10⁷ of the bone marrow cells are transferred to the mouse subject. More preferably, about 5×10⁵ to 5×10⁶ of the bone marrow cells are transferred to the mouse subject. In one specific example, about 1×10⁶ bone marrow cells are sufficient to provide a protective and/or therapeutic effect in the mouse subject.

As would be appreciated, in the case when the mouse subject has different gene background from the Eklf (K74R) mouse, e.g., different strains, then the method further comprises the step of exposing the mouse subject to a gamma irradiation or administering to the mouse subject an immunosuppressant prior to step (b). According to one embodiment, the mouse subject is exposed to a gamma irradiation of 10 Gy to suppress or reduce the immune response of the mouse subject against the modified bone marrow cells. According to another embodiment, the mouse subject is exposed to a gamma irradiation of 5 Gy for the purpose of suppressing/reducing immune response. According to still another embodiment, a gamma irradiation of 2.5 Gy is sufficient to achieve the immune-suppressive purpose.

II-(3). Methods of Preventing and/or Treating ALS via Bone Marrow Cells

The third aspect of the present disclosure is directed to a method of preventing and/or treating ALS in a subject, e.g., a human, a mouse, a rat, a chimpanzee, a rhesus monkey, a dog or a cattle. The method comprises administering to the subject an effective amount of modified bone marrow cells comprising a modified Eklf gene, which encodes a modified EKLF polypeptide comprising an amino acid modification that confers reduced sumoylation in a wild-type EKLF polypeptide.

In practice, bone marrow cells are first isolated and modified to comprise the modified Eklf gene. Depending on intended purposes, the bone marrow cells may be derived from the subject being treated/administered (i.e., autologous bone marrow cells), another subject of the same species (i.e., allogeneic bone marrow cells), or a subject of different species (i.e., xenogeneic bone marrow cells). The methods suitable for isolating/preparing bone marrow cells are known by a person having ordinary skill in the art. For example, bone marrow cells may be obtained from a human subject via bone marrow aspiration or bone marrow biopsy. Alternatively, the bone marrow cells may be obtained from a non-human subject (e.g., a mouse) by flushing method, centrifugation method, enzyme digestion, or a combination thereof. Preferably, the bone marrow cells comprises bone marrow mononuclear cells (BMMNCs), e.g., HSCs and/or HSPCs.

Then, the Eklf gene of the bone marrow cells are modified. The methods for modifying target genes (e.g., the Eklf gene) are known by a skilled artisan, e.g., site-directed mutagenesis (also known as site-specific mutagenesis or oligonucleotide-directed mutagenesis), and homologous recombination. According to some embodiments of the present discourse, the Eklf gene is modified by homologous recombination.

The thus-obtained modified Eklf gene encodes a modified EKLF polypeptide as described in Section II-(1) of the present disclosure. According to certain embodiments, the subject is a human, in which the modified EKLF polypeptide comprises the amino acid sequence of SEQ ID NO: 10, which, as compared to the wild-type human EKLF polypeptide of SEQ ID NO: 3, has a substitution of lysine residue with arginine residue at residue 54 (i.e., a K54R substitution). According to certain embodiments, the subject is a mouse, in which the modified EKLF polypeptide comprises the amino acid sequence of SEQ ID NO: 11, which, as compared to the wild-type mouse EKLF polypeptide of SEQ ID NO: 1, has a substitution of lysine residue with arginine residue at residue 74 (i.e., a K74R substitution).

As would be appreciated, the modified bone marrow cells may be administered to the subject via any appropriate route, for example, via intravenous, intraperitoneal, intraarterial or intraspinal infection. According to certain examples of the present disclosure, the modified bone marrow cells are intravenously administered to the subject.

According to certain embodiments, the subject is a mouse, in which about 1×10⁴ to 1×10⁸ of the modified bone marrow cells are transferred to the subject. Preferably, about 1×10⁵ to 1×10⁷ of the modified bone marrow cells are transferred to the subject. More preferably, about 5×10⁵ to 5×10⁶ of the bone marrow cells are transferred to the mouse subject. In one specific example, about 1×10⁶ bone marrow cells are sufficient to provide a protective and/or therapeutic effect in the mouse subject.

In general, 1×10⁷ to 1×10⁸ bone marrow cells/Kg body weight of the subject per transplant dose are required for human BMT. The minimum number is 1×10⁷ to 2×10⁷ cells/Kg body weight of the subject per transplant dose. According to some embodiments of the present disclosure, partial (20%-50%) blood replacement with the present modified bone marrow cells is sufficient to provide a therapeutic effect (e.g., alleviating or ameliorating the symptoms associated with ALS) in the recipient. In other words, 20%-50% of blood replacement (e.g., 2×10⁶ to 5×10⁷ of the present modified bone marrow cells/Kg body weight of the subject per transplant dose) is sufficient to provide a therapeutic effect in ALS patients that greatly decreases the cell number needed to be transferred and the exposed irradiation dosage, and accordingly reducing the side-effects caused by BMT and gamma irradiation. As could be appreciated, the number of the modified bone marrow cells transferred into the human subject may vary with clinical factors, such as age, gender, underlying diseases, treatment plan, hemoglobin, serum albumin, Kamofsky performance status, conditioning regimen and infection. A skilled artisan or medical practitioner may adjust or optimize the transferred number of the modified bone marrow cells in accordance with desired purposes.

According to some embodiments, the administration of the modified bone marrow cells improves the exercise behavior and learning memory in the subject.

In the case when the bone marrow cells are allogeneic or xenogeneic to the subject, the method further comprises the step of administering to the subject an immunosuppressive treatment prior to, concurrently with, or after the administration of modified bone marrow cells, so as to suppress the immune response of the subject against the allogeneic or xenogeneic bone marrow cells. The immunosuppression may be achieved by any agent and/or method known by a skilled artisan to prevent graft rejection, for example, the administration of gamma irradiation or immunosuppressant. According to some embodiments of the present disclosure, the immunosuppressive treatment is administered to the subject prior to the administration of modified bone marrow cells.

Depending on desired purposes, the immunosuppressant may be a glucocorticoid (e.g., prednisone, budesonide, prednisolone, dexamethasone or hydrocortisone), janus kinase inhibitor (e.g., tofacitinib), calcineurin inhibitor (e.g, cyclosporine or tacrolimus), mTOR inhibitor (e.g., sirolimus or everolimus), inhibitor of inosine monophosphate dehydrogenase (IMDH inhibitor; e.g., azathioprine, leflunomide or mycophenolate), biologics or monoclonal antibody (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab or daclizumab), or any agent known to suppress or reduce the immune response, such as methotrexate or mercaptopurine. A clinical practitioner or a skilled artisan may determine the type of immunosuppressant and treatment regimen in accordance with the physical conditions of the subject.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Generation of Eklf (K74R) Mice

The Eklf (K74R) mice in the C57BL/6 (B6) background were established. In brief. the K74R mutation was introduced by homologous recombination into exon 2 (E2) of the Eklf gene of B6 mice by means of a recombinant retrovirus containing the construct loxP-PGK-gb2-neo-loxP-E2 (K74R), before excising the neomycin (neo) selection marker by crossing with Ella-Cre mice. The heterozygous Eklf (K74R/+) mice were then crossed to obtain homozygous mutant Eklf (K74R/K74R) mice, hereafter termed Eklf (K74R) mice.

### Generation of ALS (N390D/+) Mice

Standard procedures were followed to generate mouse line carrying different Tardbp (TDP-43) mutations. The targeting vector carrying a mutation (N390D) on exon 6 of Tardbp was cloned in the BAC clone RP23-364 M1 by using the counter-selection BAC modification kit. The nucleotide A at position 1168 was substituted for G for N390D. A neo-resistant gene with loxP sequence cassette (PGK-neo cassette) was inserted into intron 4 of Tardbp for ES cell screening. Two independent targeted ES cell clones (#108 and #361 for N390D) were expanded and microinjected into C57BL/6 J blastocysts to generate the chimeric mice. Knock-in ES cells carrying N390D substitution in TDP-43 were identified. To remove the PGK-neo cassette from targeted Tardbp allele, the germline-transmitting F1 lines were crossed with Ella-Cre mice (Tg (Ella-cre) C5379Lmgd) expressing the Cre recombinase in the whole body. The genotypes of A315T/+ or N390D/+ mice were verified by sequencing cDNAs and genomic DNAs. The diseased mice were taken care of by the staff members including the feeding with soft food, spraying drinking water on the wall of cages, using soft materials for disable mice, etc. The knock-in mice were genotyped by PCR using the forward primer 5′-GACCTCAACTGCTCTGCTTCTACC-3′ (SEQ ID NO: 12) and the reverse primer 5′-AACGGAATCAATCCTCTCCAGG-3 (SEQ ID NO: 13).

### Generation of N390D/+/Eklf (K74R) Mice

The Eklf (K74R) mice were crossed to the ALS (N390D/+) mice so as to generate the N390D/+/Eklf (K74R) mice. All animals were maintained in a specific pathogen-free (SPF) environment under standard laboratory conditions and handled following the guidelines of the Institute Animal Care and Use Committee (IACUC) of Academia Sinica.

### Bone Marrow Transplantation (BMT)

The bone marrow cells derived from 3.5 month-old CD45.1 donor mice were transplanted to 3.5 month-old CD45 2 recipient ALS mice. Specifically, Eklf (K74R) donor mice (CD45.1) were sacrificed and their femurs were removed. Bone marrow cells were harvested by flashing the femurs with RPM 11640 medium using a 27-gauge needle and syringe. The cells were then incubated at 37° C. for 30 minutes in murine complement buffer containing antibodies against B cells, T cells and NK cells, washed twice with PBS, and then subjected to FICOLL® gradient centrifugation to collect bone marrow mononuclear cells (BMMNCs). BMMNCs (1×10⁶ cells/mouse) from donor mice were injected into the tail veins of recipient ALS mice (CD45.2) that had been exposed to total body γ-irradiation of 10, 5 or 2.5 Gy. After 12 weeks of BMT, blood constituents of CD45.1 and CD45.2 cells in the recipient ALS mice were analyzed by flow cytometry, and the exercise capacity of mice were measured by rotarod test and water maze.

### Statistical Analysis

Data are shown as mean±standard deviation (SD) or standard error of the mean (SEM). Comparisons of data under different experimental conditions were carried out using software. Each error bar represents SEM unless otherwise indicated. Significant differences in exercise ability of mice were assessed by Student's t test. A difference between groups was considered statistically significant when the p value was lower than 0.05.

### Example 1 Effect of Eklf (K74R) Mutation on ALS

Two animal models (including the genetic-crossed model and BMT model) were used to examine the effect of Eklf (K74R) mutation on ALS.

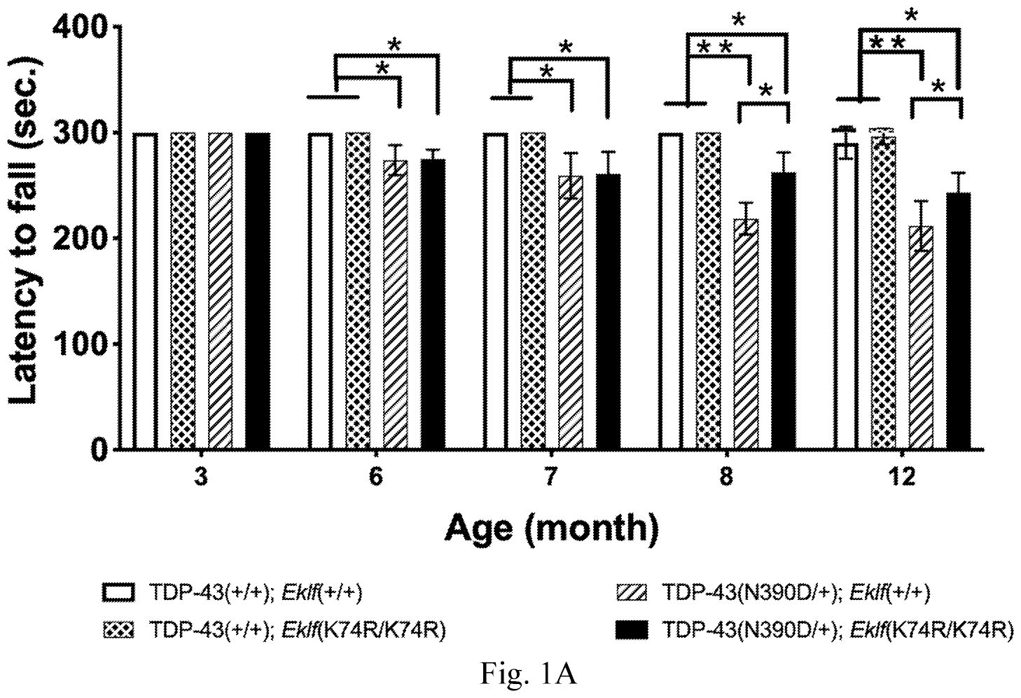

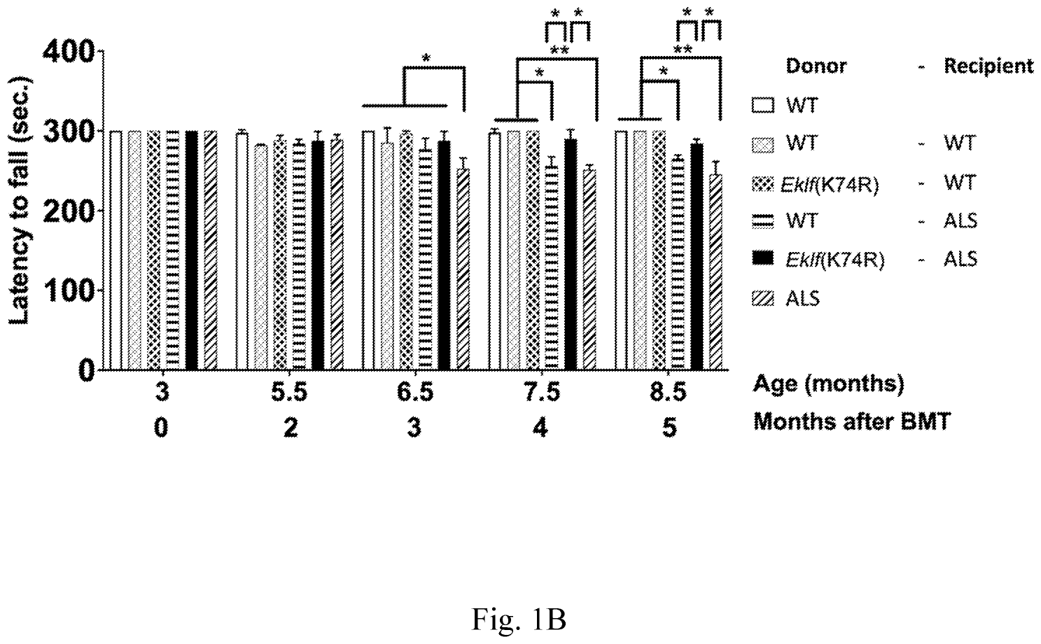

In the genetic-crossed model, the behavioral ability of N390D/+/Eklf (K74R) mice (i.e., the ALS mice having an Eklf (K74R) allele) in rotarod test was compared to that of the ALS control mice (i.e., N390D/+mice). As the data depicted in FIG. 1A, although the onset time the motor dysfunction of N390D/+/Eklf (K74R) mice was similar to that of the ALS control group, the behavioral ability of the N390D/+/Eklf (K74R) mice was significantly better than that of N390D/+ mice at eight months of age.

In the BMT model, BMMNCs from Eklf (K74R) donor mice were intravenously administered to the ALS recipient mice. The data of FIG. 1B indicated that 4 months post-BMT, the ALS mice receiving BMT from the Eklf (K74R) exhibited significantly better exercise ability than the control group.

In conclusion, the data of the present disclosure demonstrated the effect of Eklf (K74R) mutation on treating ALS. Based on the discovery, the present disclosure provides methods of treating ALS by using an Eklf (74R) nucleic acid, a EKLF (K74) polypeptide, or bone marrow cells comprising the Eklf (K74R) gene thereby alleviating or ameliorating the symptoms associated with ALS.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of preventing and/or treating a neurodegenerative disease in a subject, comprising administering to the subject an effective amount of a modified Erythroid Kruppel-like factor (EKLF) polypeptide, a modified nucleic acid encoding the modified EKLF polypeptide, or modified bone marrow cells comprising the modified nucleic acid, wherein the modified EKLF polypeptide comprises an amino acid modification that confers reduced sumoylation in a wild-type EKLF polypeptide.

2. The method of claim 1, wherein the subject is a human, and the amino acid modification is a substitution of lysine residue with arginine residue at the amino acid position 54 of a wild-type EKLF polypeptide of SEQ ID NO: 3.

3. The method of claim 2, wherein the modified EKLF polypeptide comprises the amino acid sequence of SEQ ID NO: 10.

4. The method of claim 1, wherein the subject is a mouse, and the amino acid modification is a substitution of lysine residue with arginine residue at the amino acid position 74 of a wild-type EKLF polypeptide of SEQ ID NO: 1.

5. The method of claim 4, wherein the modified EKLF polypeptide comprises the amino acid sequence of SEQ ID NO: 11.

6. The method of claim 1, wherein the modified bone marrow cells comprise modified hematopoietic stem cells (HSCs) or modified hematopoietic stem and progenitor cells (HSPCs).

7. The method of claim 1, wherein the modified bone marrow cells are autologous to the subject.

8. The method of claim 1, wherein the modified bone marrow cells are allogeneic or xenogeneic to the subject.

9. The method of claim 8, further comprising the step of exposing the subject to a gamma irradiation or administering to the subject an immunosuppressant prior to the administration of the modified bone marrow cells.

10. The method of claim 1, wherein the modified bone marrow cells are intravenously administered to the subject.

11. The method of claim 1, wherein the neurodegenerative disease is amyotrophic lateral sclerosis (ALS), Lewy Body Dementia (LBD), Parkinson's disease (PD), Alzheimer's disease (AD), multiple sclerosis (MS), or Huntington's disease (HD).


SEQUENCE LISTING (13 sequences)

SEQ ID NO: 1 - protein, 376 aa, Mus musculus
MRQKRERRPEVQGGHQPAMASAETVLPSISTLTTLGQFLDTQEDFLKWWRSEETQDLGPGPPNPTGPSHHVSLKSEDPSGEDDERDVTCAWDPDLFLTNFPGSESPGTSRTCALAPSVGPVAQFEPPESLGAYAGGPGLVTGPLGSEEHTSWAHPTPRPPAPEPFVAPALAPGLAPKAQPSYSDSRAGSVGGFFPRAGLAVPAAPGAPYGLLSGYPALYPAPQYQGHFQLFRGLAAPSAGPTAPPSFLNCLGPGTVATELGATAIAGDAGLSPGTAPPKRSRRTLAPKRQAAHTCGHEGCGKSYTKSSHLKAHLRTHTGEKPYACSWDGCDWRFARSDELTRHYRKHTGHRPFCCGLCPRAFSRSDHLALHMKRHL

SEQ ID NO: 2 - protein, 376 aa, Rattus norvegicus
MRQKRERRPEAQGGQQPVMASAETVLPSISTLTTLGQFLDTQEDFLKWWRSEETQDLGPGPPNPTEPSFHVSLKSEDPPGEDDERDVTCAWDPDLFLTNFSGPEPPSTPRTCALAPSGGPVAQFAPPESLSAYAGGPGLVTGPLGSEEHAGWAHPTPRPPTPEPFVAPVLAPGLAPKAQPAFSESRAVSAGGFFPRAGLAVPAAPSAPYGLLSGYPALYPAPQYQGHFQLFRGLAAPSAGPTAPPSFLNCLGPGAVGTELGATAIAGDAGLSPGAAPPKRSRRTLAQKRQAAHTCGHEGCGKSYTKSSHLKAHLRTHTGEKPYACSWDGCNWRFARSDELTRHYRKHTGHRPFCCGLCPRAFSRSDHLALHMKRHL

SEQ ID NO: 3 - protein, 362 aa, Homo sapiens
MATAETALPSISTLTALGPFPDTQDDFLKWWRSEEAQDMGPGPPDPTEPPLHVKSEDQPGEEEDDERGADATWDLDLLLTNFSGPEPGGAPQTCALAPSEASGAQYPPPPETLGAYAGGPGLVAGLLGSEDHSGWVRPALRARAPDAFVGPALAPAPAPEPKALALQPVYPGPGAGSSGGYFPRTGLSVPAASGAPYGLLSGYPAMYPAPQYQGHFQLFRGLQGPAPGPATSPSFLSCLGPGTVGTGLGGTAEDPGVIAETAPSKRGRRSWARKRQAAHTCAHPGCGKSYTKSSHLKAHLRTHTGEKPYACTWEGCGWRFARSDELTRHYRKHTGQRPFRCQLCPRAFSRSDHLALHMKRHL

SEQ ID NO: 4 - protein, 362 aa, Pan troglodytes [sequence omitted]

SEQ ID NO: 5 - protein, 362 aa, Macaca mulatta
MATAKTALPSISTLTALGPFPDTQDDFLKWWRSEEAQDMGPGPPDPTEPPLHVKSEDQPGEEEDDERGADATWDLDLFLTNFSGPEPGGAPQTCALAPSEAPGVQYPPPPETLGAYAGGPGMVAGLLGSEDHSGWVRPALRAPAPDAFVGPALAPAPAPEPKALALQPVYPGPGAGSSGGYFQRTGLSVPAASGAPYGLLSGYPAVYPAPQYQGHFQLFRGLQGPAPGPATSPSFLSCLGPGTVGTGLGGTAGDPGVIAEAAPSKRGRRSWARKRQAAHTCAHPGCGKSYTKSSHLKAHLRTHTGEKPYACTWEGCGWRFARSDELTRHYRKHTGQRPFRCQLCPRAFSRSDHLALHMKRHL

SEQ ID NO: 6 - protein, 369 aa, Canis lupus
MATAETALPSISTLTTLGPFPDTQEDFLKWWRSEEVQDLGPGPPDPTGLPLHVRPESQDAPGEDEDDERDSATAWDLDLLLTNFPCPEPGGAPQTCALAPGECSGVQFPPPPLPPPQAPETPCPYVGGPGLVAGLLGPEEHPGWARPAPRAPVPDAFVGSALGPAPEPKPLPLQPVYPGPGAGSSGSYFSRTGLSVPTAPGAPYGLLPGYPPLYPVPQYQGHFQLFRGLPAPAPGPTSPPSFLSCLGPGTAGSGLRGTAGDPGGVADAAPSKRSRRSWARKRQAAHTCTHPGCGKSYTKSSHLKAHLRTHTGEKPYACTWDGCGWRFARSDELTRHYRKHTGQRPFRCQLCPRAFSRSDHLALHMKRHL

SEQ ID NO: 7 - protein, 372 aa, Bos taurus
MAAAETALPSISTLTALGPFSDTQEDILKWWRSEDVQDLGPGPPDHTGPPLHVRPELEDAPGEDEDDDRDAATPWDLDLLFTNFPCPEPGGAPQTCAPQTCAPQACALAPSEGSGAQFPPPPETLGAYAGGPGLVAGLLGPEEHLGWARPAPRTPAPDTFVGPSLIPTPEPKALPLQPLYPGPGSGSSGSYFPRTGLSVPAAPGAPYGLLSGYPALYPMPQYQGHFQLFRGLQAPAPGPPSPHSFLSCLGPGTTGAGLGGTTGDPGGTTEAAPSKRSRRSWARKRQAAHTCTHPGCGKSYTKSSHLKAHLRTHTGEKPYACTWDGCGWRFARSDELTRHYRKHTGQRPFRCQLCSRAFSRSDHLALHMKRHL

SEQ ID NO: 8 - DNA, 2782 bp, Homo sapiens
  source 1..2782: mol_type = genomic DNA
tcagagttcacgaggcagccgaggaagaggaggcttgaggcccagggtgggcaccagccagccatggccacagccgagaccgccttgccctccatcagcacactgaccgccctgggccccttcccggacacacaggatgacttcctcaaggtggggcctagaaggtggggtctaggtgggctggctggaatccagggccacagtcacagatcttggggtccagacctgcatcttgacctgaaatcaagagacttaaccaggactgaggtacgctcagtccaggagaggagatctcagcttagtctggcagggggtgaggagggtggtctaggggtttgaggttctaagtgtgatctatttcggtaatagaaaacgaaggtagcctgggcaacatggtgaaaccctatctctacaaaaaataccaaaaacattaggccaggcatgggggcgtgtgcctgtagtcccaggtactccgtaggctgatgcaggaggatcattagagcccaggagattaaggatacagtgagctgcaccactgcactccagcctgggcaaaagagtaagaccctatctcaagaaaaaaaaaaaaaaaaaggaacgagatctaggctcacagacaatcttccagatatcagcgggaaatgatgagtgtgtctgggggacatccaaaatttcggaattaatcttgttttgggagacagggaaggagagggatgttctgggggaaaactaagtcaaggctggcatcctctcccccgtcccctgccagttttccatctccagcagctctgctaccccttcccccatccccgagtgtggttccagatagtggaagtcttatctcctgtctccagccagacctgatcggtttctgtccctggagctggggggggagcggggagaggggcggttagaggggcagtgttggggaagtgggacagacagacaggcaaacaagacccctttccaaagcctctgcgtcagagtgtccagcccgcgatgtccctgggcagggcaccccagtgtccaccgaacctcgagctgcctgctccctcccgcagtggtggcgctccgaagaggcgcaggacatgggcccgggtcctcctgaccccacggagccgcccctccacgtgaagtctgaggaccagcccggggaggaagaggacgatgagaggggcgcggacgccacctgggacctggatctcctcctcaccaacttctcgggcccggagcccggtggcgcgccccagacctgcgctctggcgcccagcgaggcctccggggcgcaatatccgccgccgcccgagactctgggcgcatatgctggcggcccggggctggtggctgggcttttgggttcggaggatcactcgggttgggtgcgccctgccctgcgagcccgggctcccgacgccttcgtgggcccagccctggctccagccccggcccccgagcccaaggcgctggcgctgcaaccggtgtacccggggcccggcgccggctcctcgggtggctacttcccgcggaccgggctttcagtgcctgcggcgtcgggcgccccctacgggctactgtccgggtaccccgcgatgtacccggcgcctcagtaccaagggcacttccagctcttccgcgggctccagggacccgcgcccggtcccgccacgtccccctccttcctgagttgtttgggacccgggacggtgggcactggactcggggggactgcagaggatccaggtgtgatagccgagaccgcgccatccaagcgaggccgacgttcgtgggcgcgcaagaggcaggcagcgcacacgtgcgcgcacccgggttgcggcaagagctacaccaagagctcccacctgaaggcgcatctgcgcacgcacacaggtgagggggcggggccccggacatgagaaagggcgcggcgcccgctgtagttacaggggaagaagggttgcagagggcgggacttggacttggctggcctctgagagtgagtgcctccttaaattttgtgccctaggggcctcactttgttcatcctagtcccagcccaggctgagtaaaggggtgtggccagatgcaggggacccggggacatgactgggcagacagtggcgcttatggcttccttgtcccctaggggagaagccatacgcctgcacgtgggaaggctgcggctggagattcgcgcgctcggacgagctgacccgccactaccggaaacacacggggcagcgccccttccgctgccagctctgcccacgtgctttttcgcgctctgaccacctggccttgcacatgaagcgccacctttgagccctgccctggcacttggactctcctagtgactggggatgggacaagaagcctgtttggtggtctcttcacacggacgcgcgtgacacaatgctgggtggttttcccacgaatggaccctctcctggactcgcgttcccaaagatccacccaaatatcaaacacggacccatagacagccctgggggagcctcttacggaaaatccgacaagccttcagccacagggagccacacagagatgtccaaactgtcgtgcaaacccagtgagacagaccgccaaataaacggactcagtggacactcagaccagctcccagatggccctggacagcaggagagggtgtgggatgaggcttcccagagaccctgggtctagaaagcggctcctgaaggtcccttattgtggctgatattaactgtcaatggttatgggtcctataaaaatgcccctcccagataaa

SEQ ID NO: 9 - DNA, 3368 bp, Mus musculus
  source 1..3368: mol_type = genomic DNA
gtgggcagacaggagccctccaagaaactttcctagcctcatagcccatgaggcagaagagagagaggaggcctgaggtccagggtggacaccagccagccatggcctcagctgagactgtcttaccctccatcagtacactcaccaccctgggacagttcctggacacccaggaggacttcctcaaggtggggccagtgtgagtgtgtgggagggggcaggtggtcttgcatagggcatagtgcttaggggtggggcgtctatcttactttaatatcctctgctctgttttttgggggtggaggagtgggagagcctctgagccttgtttgggggagatgttctaggggtctgagatcaaggtgaggcctatttctccaacaggaagcagaattctaagctctatatcttaagagactaggctcatagagacaaaggtccagataaaggtgtcctgggatttccaggctttgagctgtaattttctgggctatgtgaagacagggaaaggctagggaaaacggagtcgaagctgtcccctttgactcagaactctgcaaccccttctcccatcctgaatactattcttggtaagtgtcttagctgtctctagcaagacctaatggagttgtctggagctgagaaaggggttaggggaaccgtgtgggtaaatgacaggcaccaacggtgtttccagccagggttgtttgagggccaggtacccagtgcctaccattcaagcagtacgctccctcccgcagtggtggcggtctgaggagacgcaggatttggggccggggcccccgaatcccacggggccgtcccatcacgtgagtctgaaatcggaggacccttccggagaggacgatgagagggacgtgacctgtgcgtgggacccggatcttttccttacaaactttccaggttccgagtctcccggcacttcccggacctgtgccctggcgcccagcgtggggccagtggcacagttcgagccgcctgagtctctgggcgcctatgcgggtggcccagggttggtgactgggcctttgggctccgaggagcacacaagctgggcgcacccgactccgagacccccagcccctgaacccttcgtggcccctgccctggccccgggactcgctcccaaggctcagccctcgtactccgactcgcgagcgggctccgtagggggcttcttcccgcgggcggggcttgcggtgcccgcagctccaggcgccccctatgggctgctgtcgggataccccgcgctgtaccccgcgccacagtaccaaggccacttccagctctttcgcgggctcgcggcgccttctgctggtcccacggcgcccccttccttcttgaattgtctgggacctgggactgtggccacagaactcggggccactgcgatcgccggagacgcaggcttgtccccgggaactgcgccgcccaaacgcagccggcgaactttggcacctaagaggcaggcggcacatacgtgcgggcacgaaggctgcgggaagagctacaccaagagctcgcacctcaaggcgcacctgcgcacgcacacgggtaagggcggggccagacgggcgggggcggggcgggagccgctagtgaacgaagggaggggccggagggtagtcagaggcgtggctaaaggcggccccagttctaggggtcgtgaagaccgcacctgagacactgggtcaagtctagaaggggcgattccagacccaaatgggctaatacaaacactcgggaggcagaggcaggtggatagcagtgacttcgaggccatttgggctattatagcgagtttcagcagcctgagctacttagtgagatcctggttcataaataaataggtgtaacagaggacctggggaacactttggggacttcggtgttagaagtggatgtgtaaggcctgggttagagatgggagaagaaactagaggggtgaacccgaaaggtacaagcttggaatgccagagctcaggatatagccagtatttacatgcatgctcgagctggaaccatctgggatcaggaggttgagacactcaagtaaaatcagtttcaggggcaactgacagaggtcccagagttaagaaaagaagagaagggggctggagagatggctcagtggttaagagcactgactgctcttccagaggtcctgagtttaaatcccagcaccacatggtggctcacaaccatctgtaatgggatccgatgccctcttctggtgtgactgaagacagctacagtgtacttacatataataaataaataaattaaaaaaaaaaagaaagaaagaaaaaagaaaagaagagaaggaaatgctgagagacagggcctagaaagagaaacggggtcatcccaggactggaagacagctgagggtctcccaagcatggcagggcacgcaacaggctgtaacaggaagagagggaatcaccagagacagggccttgaacactggggtggatttctgggcttgaaccaagttgaggaacaagactggatatcatcgggaggctctgccagagcaagaaatagctgcaacgcggagaacaaagaacgaaggtgcagccacataaaaaggcagggaactagcacaccggaagtgggataggagaccggaagtgagaaaactgcaggattgcagctgtagatacagaaaaggattgagtcacagaaggcaggattatgtgaccttttaactgtgtgggctaggtatgtcctaagacttggctctacttcatcaagggtgcaaactggagctgggttgcttggagggtggtacttacagctccctgtccttcaggagagaagccttatgcctgctcctgggacggctgtgactggaggttcgctcgctcagacgaactgacgcgccactaccggaagcacactggacatcgtcccttctgctgtggcctctgcccacgtgctttttcacgctctgaccacttagctctgcacatgaagcgtcacctctgagtgatcctgcacaaggactggggatgaaataagagtggatccaaggaccgtatcccaaaagatgggccattatatagtcctacccagatcaaaaactgaccagaagaccatacaaaggagccttcaggacaaacctcacatgtcctcagggagccccacacatggccccacagacccagcaatatagaccaccagataaatcaactcaaatggacccctagaccagaggagtgaccctgtgtcctggacgcagatggactggggtgagatttcctaagatctagaagggagcttcacactgtgcccatctgctaggattgttgtcgttactataaaaatttcccatataaaaccag

SEQ ID NO: 10 - protein, 362 aa, Homo sapiens
MATAETALPSISTLTALGPFPDTQDDFLKWWRSEEAQDMGPGPPDPTEPPLHVRSEDQPGEEEDDERGADATWDLDLLLTNFSGPEPGGAPQTCALAPSEASGAQYPPPPETLGAYAGGPGLVAGLLGSEDHSGWVRPALRARAPDAFVGPALAPAPAPEPKALALQPVYPGPGAGSSGGYFPRTGLSVPAASGAPYGLLSGYPAMYPAPQYQGHFQLFRGLQGPAPGPATSPSFLSCLGPGTVGTGLGGTAEDPGVIAETAPSKRGRRSWARKRQAAHTCAHPGCGKSYTKSSHLKAHLRTHTGEKPYACTWEGCGWRFARSDELTRHYRKHTGQRPFRCQLCPRAFSRSDHLALHMKRHL

SEQ ID NO: 11 - protein, 376 aa, Mus musculus
MRQKRERRPEVQGGHQPAMASAETVLPSISTLTTLGQFLDTQEDFLKWWRSEETQDLGPGPPNPTGPSHHVSLRSEDPSGEDDERDVTCAWDPDLFLTNFPGSESPGTSRTCALAPSVGPVAQFEPPESLGAYAGGPGLVTGPLGSEEHTSWAHPTPRPPAPEPFVAPALAPGLAPKAQPSYSDSRAGSVGGFFPRAGLAVPAAPGAPYGLLSGYPALYPAPQYQGHFQLFRGLAAPSAGPTAPPSFLNCLGPGTVATELGATAIAGDAGLSPGTAPPKRSRRTLAPKRQAAHTCGHEGCGKSYTKSSHLKAHLRTHTGEKPYACSWDGCDWRFARSDELTRHYRKHTGHRPFCCGLCPRAFSRSDHLALHMKRHL

SEQ ID NO: 12 - DNA, 24 bp, synthetic construct
  misc_feature 1..24: note = Forward primer
  source 1..24: mol_type = other DNA
gacctcaactgctctgcttctacc

SEQ ID NO: 13 - DNA, 22 bp, synthetic construct
  misc_feature 1..22: note = Reverse primer
  source 1..22: mol_type = other DNA
aacggaatcaatcctctccagg